(19) United States
(12) Patent Application Publication
(10) Pub. No.: US 2015/0141978 A1
(43) Pub. Date: May 21, 2015

(54) ABLATION MEDICAL DEVICES AND METHODS FOR MAKING AND USING ABLATION MEDICAL DEVICES

(71) Applicant: BOSTON SCIENTIFIC SCIMED, INC., Maple Grove, MN (US)

(72) Inventors: RAJ SUBRAMANIAM, FREMONT, CA (US); PAUL A. ROCHE, SAN JOSE, CA (US)

(73) Assignee: BOSTON SCIENTIFIC SCIMED, INC., Maple Grove, MN (US)

(21) Appl. No.: 14/548,007

(22) Filed: Nov. 19, 2014

Related U.S. Application Data
(60) Provisional application No. 61/906,789, filed on Nov. 20, 2013.

(51) Int. Cl. A61B 18/14; A61B 18/12

(52) U.S. Cl. CPC A61B 18/1492; A61B 18/1206; A61B 2018/00577
USPC 606/34

(57) ABSTRACT

Ablation devices and methods for determining a degree of contact between an electrode and a target tissue are disclosed. An example ablation device for treating body tissue may include a catheter having a proximal end region and a distal end region. An electrode may be disposed adjacent to the distal end region of the catheter. The device may also include a processing unit having a memory. The processing unit may be in electrical communication with the electrode. The processing unit may be capable of determining a degree of contact between the electrode and a target tissue.

### CROSS-REFERENCE TO RELATED APPLICATIONS

This application claims priority under 35 U.S.C. §119 to U.S. Provisional Application Ser. No. 61/906,789, filed Nov. 20, 2013, the entire disclosures of which are incorporated herein by reference.

### TECHNICAL FIELD

The present disclosure relates generally to a method for monitoring ablation. More specifically, the present disclosure pertains to a method for predicting the degree of contact between an ablation electrode and tissue.

### BACKGROUND

The treatment of cardiac arrhythmias is sometimes performed in conjunction with an ablation catheter inserted into a chamber of the heart or in one of the vessels leading into or from the heart. In the treatment of atrial fibrillation, for example, a radio frequency (RF) ablation catheter equipped with a number of electrodes can be brought into contact with cardiac tissue for creating one or more ablation points along the tissue. During ablation, an RF generator supplies electrical energy to the electrodes. As the RF energy from the tip electrode passes through the contacting tissue to the ground pad, heat is generated in the tissue. The resulting heat from this electric field forms a controlled lesion that blocks the electrical impulses from being conducted through the tissue and serves to promote the normal conduction of electrical impulses through the proper electrical pathway within the heart. The effectiveness of the lesions created by RF ablation can vary significantly depending on how well the energy is transferred to the tissue from the ablation electrode even when the same RF power and ablation durations are used. Several factors influence this energy transfer.

### SUMMARY

This disclosure provides design, material, manufacturing method, and use alternatives for medical devices. An example medical device may include an ablation device for treating body tissue, which may include a catheter having a proximal end region and a distal end region. An electrode may be disposed adjacent to the distal end region of the catheter. The device may also include a processing unit and memory. The processing unit may be in electrical communication with the electrode. The processing unit may be capable of determining a degree of contact between the electrode and a target tissue.

An example method for ablating a target tissue may include advancing an ablation device through a body lumen to a position adjacent to a target tissue. The ablation device may include a catheter shaft having an ablation electrode coupled to the catheter shaft. The method may also include measuring the impedance at the target tissue at a plurality of different frequencies including a first frequency and a second frequency and determining a first or “baseline” difference between these impedance measurements. The method may also include ablating the target tissue and, during or after ablation, determining additional differences between the impedance measurements at the first frequency and at the second frequency. Finally, the method may include comparing the baseline impedance with the other measured differences in impedance so as to determine the degree of contact between the ablation electrode and the target tissue.

Another example ablation device for treating body tissue may include a catheter having a proximal end region and a distal end region. An electrode may be disposed adjacent to the distal end region of the catheter. The ablation device may also include a processing unit with memory. The processing unit may be in electrical communication with the electrode. The processing unit may be capable of determining a degree of contact between the electrode and a target tissue by monitoring the change in impedance at two different frequencies and determining if the impedance drops at the same rate at both frequencies.

An example ablation device for treating body tissue may comprise:

a catheter having a proximal end region and a distal end region;

an electrode disposed adjacent to the distal end region of the catheter; and

a processing unit having a memory, the processing unit in electrical communication with the electrode;

wherein the processing unit is capable of determining a degree of contact between the electrode and a target tissue.

Alternatively or additionally to any of the embodiments above, wherein the processing unit monitors impedance at a plurality of different frequencies.

Alternatively or additionally to any of the embodiments above, wherein the processing unit calculates the difference between the impedance monitored at a first electrode at a first frequency from the impedance monitored at the first electrode at a second frequency.

Alternatively or additionally to any of the embodiments above, wherein during an ablation procedure, the processing unit is configured to monitor the change in impedance to determine if the change in impedance follows a substantially linear relationship to determine the degree of contact between the electrode and the target tissue.

Alternatively or additionally to any of the embodiments above, wherein the electrode includes an ablation electrode.

Alternatively or additionally to any of the embodiments above, further comprising one or more mapping electrodes coupled to the catheter.

Alternatively or additionally to any of the embodiments above, wherein the processing unit includes a mapping processor.

Alternatively or additionally to any of the embodiments above, wherein the processing unit includes an RF generator.

An example method for ablating a target tissue may comprise:

advancing an ablation device through a body lumen to a position adjacent to a target tissue, the ablation device including a catheter shaft having an ablation electrode coupled to the catheter shaft;

measuring the impedance at the target tissue at a plurality of different frequencies including a first frequency and a second frequency;

determining a first difference between the impedance measured at the first frequency and the impedance measured at the second frequency;

ablating the target tissue with the ablation electrode;

during or after ablating the target tissue, determining a plurality of additional differences between the impedance measured at the first frequency and the impedance measured at the second frequency; and

comparing the first difference between the impedance measured at the first frequency and the impedance measured at the second frequency with the plurality of additional differences between the impedance measured at the first frequency and the impedance measured at the second frequency so as to determine the degree of contact between the ablation electrode and the target tissue.

Alternatively or additionally to any of the embodiments above, wherein the first frequency, the second frequency, or both is approximately 46-460 kHz.

Alternatively or additionally to any of the embodiments above, wherein the first frequency is approximately 46 kHz.

Alternatively or additionally to any of the embodiments above, wherein the second frequency is approximately 460 kHz.

Alternatively or additionally to any of the embodiments above, wherein the ablation device is coupled to a processing unit, and wherein the processing unit compares the first difference in impedance with the plurality of additional differences.

Alternatively or additionally to any of the embodiments above, wherein the processing unit includes an RF generator.

Alternatively or additionally to any of the embodiments above, wherein comparing the first difference between the impedance measured at the first frequency and the impedance measured at the second frequency with the plurality of additional differences between the impedance measured at the first frequency and the impedance measured at the second frequency so as to determine the degree of contact between the ablation electrode and the target tissue includes determining if the first difference and the plurality of differences follow a substantially linear relationship.

Alternatively or additionally to any of the embodiments above, wherein comparing the first difference between the impedance measured at the first frequency and the impedance measured at the second frequency with the plurality of additional differences between the impedance measured at the first frequency and the impedance measured at the second frequency so as to determine the degree of contact between the ablation electrode and the target tissue includes determining if the impedance drops at substantially the same rate at both the first frequency and at the second frequency.

Alternatively or additionally to any of the embodiments above, wherein the ablation device includes a display unit with a colored indication of approximate contact between the ablation electrode and the target tissue.

Alternatively or additionally to any of the embodiments above, further comprising measuring the impedance at the target tissue at a third frequency different from both the first frequency and the second frequency.

Alternatively or additionally to any of the embodiments above, wherein the target tissue is a cardiac tissue.

An example ablation device for treating body tissue may comprise:

a catheter having a proximal end region and a distal end region;

an electrode disposed adjacent to the distal end region of the catheter;

a processing unit having a memory, the processing unit in electrical communication with the electrode;

wherein the processing unit is capable of determining a degree of contact between the electrode and a target tissue by monitoring the change in impedance at two different frequencies and determining if the impedance drops at the same rate at both frequencies.

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1 is a schematic view of an ablation system in accordance with an illustrative embodiment;

FIG. 2 is graph illustrating impedance versus electrode contact; and

FIG. 3 is a graph illustrating the difference in impedance at two difference frequencies versus percent of electrode surface contact.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

For purposes of this disclosure, “proximal” refers to the end closer to the device operator during use, and “distal” refers to the end farther from the device operator during use.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

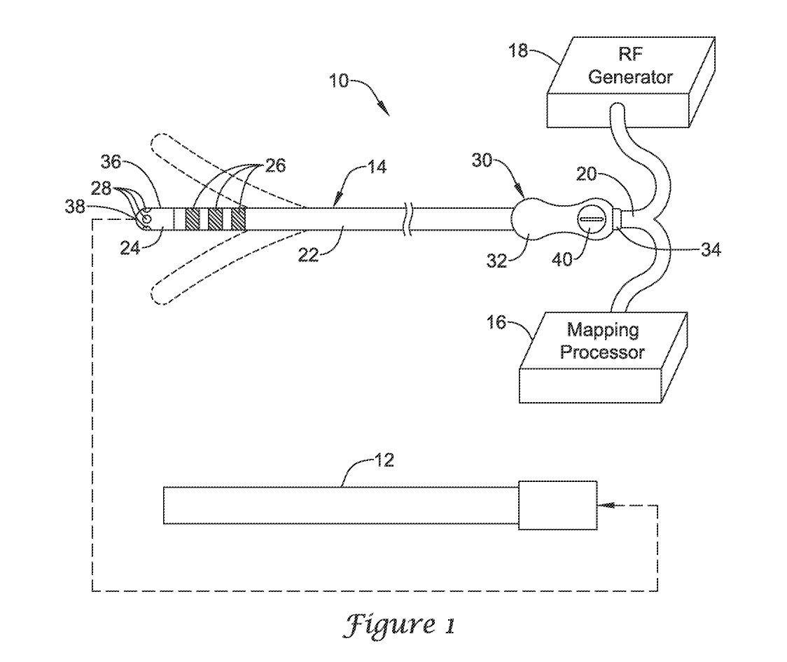

FIG. 1 is an illustrative electrophysiology system 10 for use in ablating body tissues. The system 10 may be used within body lumens, chambers or cavities of a patient for therapeutic and diagnostic purposes in those instances where access to interior bodily regions is obtained through, for example, the vascular system or alimentary canal and without complex invasive surgical procedures. For example, the system 10 has application in the diagnosis and treatment of arrhythmia conditions within the heart. The system 10 also has application in the treatment of ailments of the gastrointestinal tract, prostrate, brain, gall bladder, uterus, and other regions of the body. As an example, the system 10 will be described hereinafter for use in the heart for mapping and ablating arrhythmia substrates.

The system 10 may generally comprise a conventional guide sheath 12, and an electrophysiology catheter 14 that can be guided through a lumen (not shown) in the guide sheath 12. The catheter 14 is configured to be introduced through the vasculature of the patient, and into one of the chambers of the heart, where it can be used to map and ablate myocardial tissue. The system 10 may also include a mapping processor 16 and a source of ablation energy, and in particular, a radio frequency (RF) generator 18, coupled to the catheter 14 via a cable assembly 20. Although the mapping processor 16 and RF generator 18 are shown as discrete components, they can alternatively be incorporated into a single integrated device. In some embodiments a recorder system may also be provided as a discrete component or as an integrated device with the mapping processor and/or RF generator.

The mapping processor 16 is configured to detect, process, and record electrical signals within the heart via the catheter 14. Based on these electrical signals, a physician can identify the specific target tissue sites within the heart, and ensure that the arrhythmia causing substrates have been electrically isolated by the ablative treatment. Based on the detected electrical signals, the mapping processor 16 outputs electrocardiograms (ECGs) to a display (not shown), which can be analyzed by the user to determine the existence and/or location of arrhythmia substrates within the heart and/or determine the location of the catheter 14 within the heart. In an optional embodiment, the mapping processor 16 can generate and output an isochronal map of the detected electrical activity to the display for analysis by the user. The RF generator 18 is configured to deliver ablation energy to the catheter 14 in a controlled manner in order to ablate the target tissue sites identified by the mapping processor 16.

The mapping processor 16 and RF generator 18 may provide radio-frequency (RF) energy to electrodes (e.g., the ablation, mapping, and/or microelectrodes as disclosed herein) mounted on the catheter 14 as well as enable the user to record, view and analyze intracardiac electrogram and EKG signals, and to view a real-time graphic representation of the catheters being used. The mapping processor 16 and/or RF generator 18 may include a computer or other processing device, and memory or other storage device. Alternatively, the processing device and the storage device can be one or more separate units. In some instances, real time images and/or data may be generated and displayed on one or more displays (not explicitly shown) of the mapping processor 16 and/or RF generator 18. The system 10 may also include an input device, such as a keyboard or mouse, for programming the system 10 and/or for controlling certain functions of the system 10. These functions may include the powering up of the RF generator 18 to supply energy to one or more of the electrodes for mapping or ablating cardiac tissue, for example.

The catheter 14 may be advanced though the guide sheath 12 to the target location. The sheath 12 may be advanced over a guidewire in conventional fashion. Alternatively, a steerable sheath may be provided. A sheath introducer (not shown), such as those used in combination with basket catheters, may be used when introducing the catheter 14 into the sheath 12. The guide sheath 12 preferably includes a radio-opaque compound, such as barium, so that the guide sheath 12 can be observed using fluoroscopic or ultrasound imaging, or the like. Alternatively, a radio-opaque marker (not shown) can be placed at the distal end of the guide sheath 12.

The catheter 14 may include an integrated flexible catheter body 22, a plurality of distally mounted electrodes, and in particular, a tissue ablation electrode 24, a plurality of mapping ring electrodes 26, a plurality of mapping microelectrodes 28, and a proximally mounted handle assembly 30. In alternative embodiments, the flexible catheter 14 may be replaced with a rigid surgical probe if percutaneous introduction or introduction through a surgical opening within a patient is desired.

The handle assembly 30 may include a handle 32 composed of a durable and rigid material, such as medical grade plastic, and ergonomically molded to allow a physician to more easily manipulate the electrophysiology catheter 14. The handle assembly 30 may include an external connector 34, such as an external multiple pin connector, received in a port on the handle assembly 30 with which the cable assembly 20 mates, so that the mapping processor 16 and RF generator 18 can be functionally coupled to the catheter 14. The handle assembly 30 may further including a steering mechanism 40, which can be manipulated to bidirectionally deflect the distal end of the electrophysiology catheter 14 (shown in phantom) via steering wires (not shown).

In the illustrated embodiment, the tissue ablation electrode 24 may take the form of a cap electrode mounted to the distal tip of the catheter body 22. In particular, the ablation electrode 24 may have cylindrically-shaped proximal region 36 and a hemispherical distal region 38. The ablation electrode 24 may have any suitable length; for example, but not limited to, in the range between 4 millimeters (mm) and 10 mm. In some embodiments, the ablation electrode 24 is composed of a solid, electrically conductive material, such as platinum, gold, or stainless steel. The ablation electrode 24 is electrically coupled to the RF generator 18, so that ablation energy can be conveyed from the RF generator 18 to the ablation electrode 24 to form lesions in myocardial tissue.

The mapping ring electrodes 26 may include a three mapping ring electrodes, although any number of mapping electrodes 26 can be used. The mapping ring electrodes 26, as well as the tissue ablation electrode 24, are capable of being configured as bipolar mapping electrodes. For example, the ablation electrode 24 and each mapping ring electrode 26 can be individually combined to create three mapping pairs. In the illustrated embodiment, the mapping ring electrodes 26 may be composed of a solid, electrically conducting material, like platinum, gold, or stainless steel, attached about the catheter body 22. Alternatively, the mapping ring electrodes 26 can be formed by coating the exterior surface of the catheter body 22 with an electrically conducting material, like platinum or gold. The coating can be applied using sputtering, ion beam deposition, or equivalent techniques. The mapping ring electrodes 26 can have suitable lengths, such as, but not limited to, between 0.5 mm and 5 mm. The mapping ring electrodes 26 are electrically coupled to the mapping processor 16, so that electrical events in myocardial tissue can be sensed for the creation of electrograms or monophasic action potentials (MAPs), or alternatively, isochronal electrical activity maps.

Like the mapping ring electrodes 26, the mapping microelectrodes 28 are electrically coupled to the mapping processor 16, so that electrical events in myocardial tissue can be sensed for the creation of electrograms or MAPS, or alternatively, isochronal electrical activity maps. The microelectrodes 28 may be disposed on the tissue ablation electrode 24, and in particular, may be embedded within the wall of the tissue ablation electrode 24. This allows the localized intracardial electrical activity to be measured in real time at the point of energy delivery from the ablation electrode 24. In addition, due to their relatively small size and spacing, the microelectrodes 28 do not sense far field electrical potentials that would normally be associated with bipolar measurements taken between the tissue ablation electrode 24 and the mapping ring electrodes 26.

Instead, the microelectrodes 28 measure the highly localized electrical activity at the point of contact between the ablation electrode 24 and the endocardial tissue. Thus, the arrangement of the microelectrodes 28 may substantially enhance the mapping resolution of the electrophysiology catheter 14. The high resolution inherent in the microelectrode arrangement may allow a user to more precisely measure complex localized electrical activity, resulting in a powerful tool for diagnosing ECG activity; for example, the high frequency potentials that are encountered around pulmonary veins or the fractioned ECGs associated with atrial fibrillation triggers.

The effectiveness of the lesions created by RF ablation can vary significantly depending on how well the energy is transferred to the tissue from the ablation electrode 24, even when the same RF power and ablation durations are used. Several factors may influence this energy transfer. One variable that may impact how well energy is transferred to the target tissue from the ablation electrode 24 may be the degree of contact between the ablation electrode 24 and the tissue to be treated. Previous devices have used contact-source sensing or strain gauge sensing which may determine the amount of force with which the ablation electrode 24 contacts the tissue. However, it may be desirable to determine the degree of the electrical contact between the ablation electrode 24 and the tissue to be treated.

While the present method is discussed with reference to ablation electrode 24 and the system 10 described with respect to FIG. 1, it is contemplated that the method is applicable to various electrophysiology or ablation systems and/or electrodes, including electrodes of different shapes, sizes, and functions. For example, it is contemplated that the method described herein may be applied to ring electrodes 26 or microelectrodes 28, which may provide additional information about the catheter 14 orientation with respect to the tissue. To determine the degree of contact between the ablation electrode 24 and the target tissue, the impedance between the ablation electrode 24 and the dispersive electrode can be monitored at two different frequencies before beginning the treatment and during treatment. The difference between the measured impedance at the two frequencies may correlate to the degree of electrode to tissue contact. For each electrode configuration (size, shape, etc.) a threshold target impedance difference may be established that would ensure a significant portion of the energy input will be delivered to the target tissue. For example, for a given electrode tip size the relationship between the impedance difference and the amount of electrode contacting the tissue will be approximately the same across different patients and/or devices.

In some instances, impedance may be used to approximate the degree of contact between the ablation electrode 24 and the tissue to be treated. The impedance of the cardiac tissue is higher than that of the blood. Typically part of ablation electrode 24 is exposed to the blood while the rest of electrode is in contact with the tissue. This may be reflected in the impedance measured by the RF generator 18 between the ablation electrode 24 and a dispersive independent electrode (not explicitly shown). For example, when a portion of the ablation electrode 24 contacts the cardiac tissue and a portion of the electrode contacts blood, the measured impedance will fall in between the impedance of the cardiac tissue and impedance of the blood. In some instances, the measured impedance may be proportional to or otherwise correlated to the proportion or fraction of the electrode surface area contacting the tissue. For example, the impedance measured increases as more and more of the ablation electrode 24 tip surface is covered by or contacts the target tissue.

Thus, the greater the surface area of the ablation electrode 24 contacting the target tissue the higher the impedance may be as more current travels through the higher impedance tissue from the ablation electrode 24 to the dispersive electrode. In some instances, the dispersive electrode may be a ground patch electrode supplied on the patient's body. However, it is contemplated that the dispersive electrode may be positioned on the catheter 14.

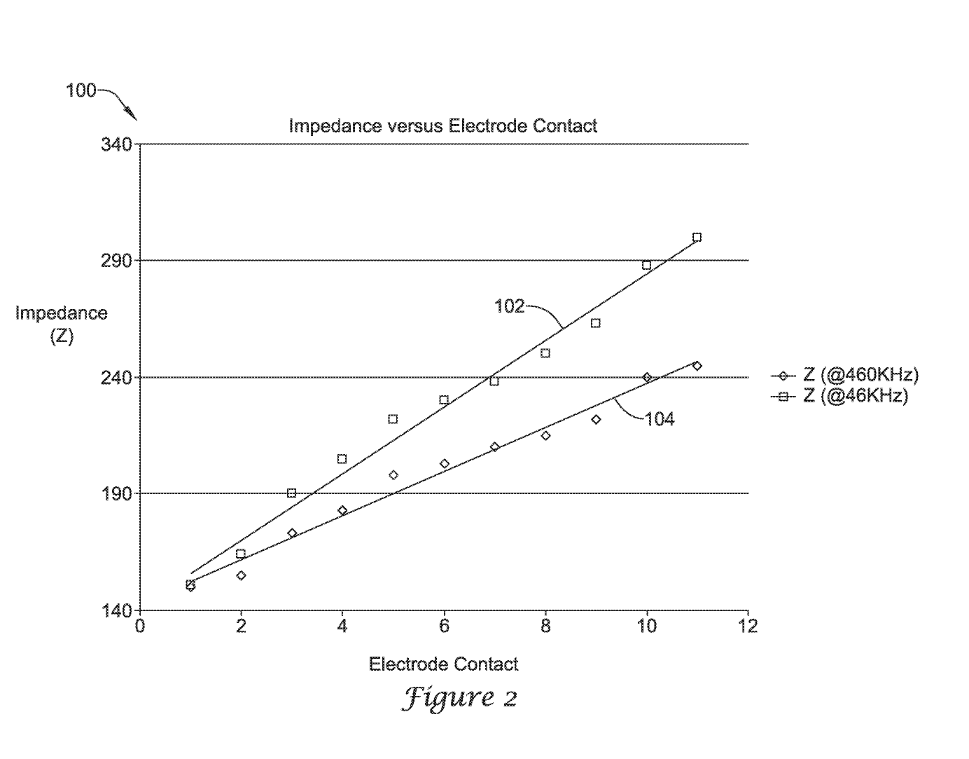

Further, the measured impedance may also be dependent on the frequency at which the impedance is measured. At lower frequencies the impedance increases relatively sharply in a fairly linear manner as the surface area of the ablation electrode 24 in contact with the tissue increases. At higher frequencies, the rise in impedance as function of electrode surface contact is less dramatic, but also fairly linear, as illustrated in FIG. 2.

FIG. 2 illustrates a graph 100 of the measured impedance (Z) versus the electrode contact for two different frequencies. As can be seen, the measured impedance at a first frequency (e.g., 46 kilohertz (kHz)) 102 rises more quickly (or has a steeper slope) than the measured impedance at a second frequency (e.g., 460 kHz) 104. The impedance measurements obtained at 460 kHz may be less sensitive to changes in the degree of ablation electrode 24 contact than the measurements obtained at 46 kHz. However, as shown in FIG. 2, for both the lower frequency and the higher frequency, the impedance increases in a fairly linear manner as the amount of the ablation electrode 24 contacting the target tissue increases. While the relationship between the measured impedance and the degree of electrode 24 contact is described with respect to impedance measurements at 46 kHz and 460 kHz, it is contemplated that other frequencies may be used as desired including about 10-1000 kHz, or about 25-600 kHz, or about 46-460 kHz, or other frequencies. In some instances, the impedance may be measured at more than two frequencies, which may improve error mitigation.

As energy is delivered to the target tissue and ablation of the tissue proceeds, the impedance begins to fall. It is contemplated that during the ablation of a target tissue, the measured impedance may drop at approximately the same rate for the measurements obtained at both a low frequency and a high frequency. It is further contemplated that the impedance may also drop if the ablation electrode 24 moves away from the target tissue reducing the surface area of the ablation electrode 24 contacting the target tissue. An increase in impedance may occur with complete desiccation of the target tissue.

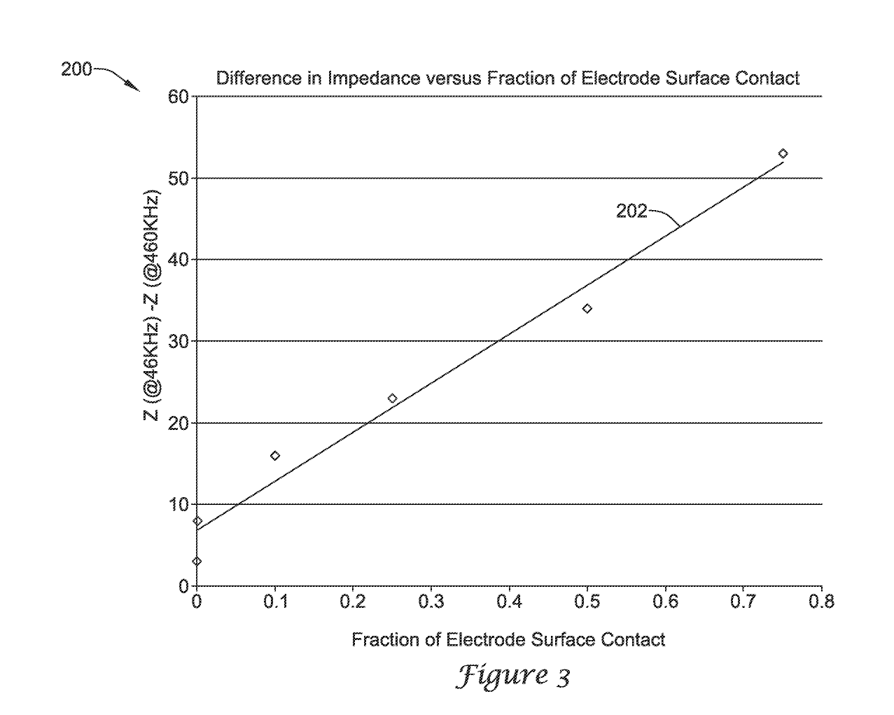

Since the measured impedance may drop at approximately the same rate for the measurements obtained at both a low frequency and a high frequency, a direct linear relationship may be present between the difference in the impedance measured at a low frequency and a high frequency to the fraction of the electrode surface contact. FIG. 3 illustrates the difference in the impedance (Z) measured at 46 kHz and the impedance measured at 460 kHz (Z_{46kHz}−Z_{460kHz}) versus the fraction of the ablation electrode 24 surface contacting the target tissue. More specifically, the difference in impedance is the measured impedance at the lower frequency (in this instance 46 kHz) minus the measured impedance at the higher frequency (in this instance 460 kHz) or Z_{46kHz}−Z_{460kHz}. As can be seen in FIG. 3, the difference between the impedance measured at a low and a high frequency has a generally linear relationship 202 with the fraction of the electrode surface contacting the target tissue. It is contemplated that the relationship 202 between the difference in measured impedance and electrode contact may be used to estimate the level of contact between the electrode surface and the target tissue in the body. The catheter 14 may be advanced intravascularly or percutaneously such that the ablation electrode 24 is adjacent to the desired treatment region. Prior to beginning the ablation procedure an initial measurement of impedance in blood (for example, with no tissue contact) may be performed prior to beginning the treatment, however this is not required. This may be used to calibrate the system 10. The ablation electrode 24 may then be brought into contact with the target tissue and the impedance measurements at a low frequency and a high frequency may begin. The clinician may apply additional force or pressure to the catheter 14 until the system indicates the electrode 24 is in good contact with the target tissue, or meets the threshold target impedance difference between the low frequency and the high frequency impedance measurements. In some embodiments, the system 10 may be configured to provide a visual and/or audio indication to the clinician indicating whether or not the electrode 24 is in good contact with the target tissue. For example, the ablation system 10 may display a colored indication of approximate electrode contact. For example, a red light may indicate poor electrode 24 to tissue contact while a green light may indicate good electrode 24 to tissue contact. It is contemplated that this may allow the clinician to use only the necessary force required which may reduce the chance of perforation by applying excessive force.

Once good electrode 24 to tissue contact has been obtained, RF energy may be supplied to the ablation electrode 24 to create the desired lesion. During the ablation procedure, a processing unit (e.g., the mapping processor 16, the RF generator 18, combinations thereof, or another component of system 10) may monitor the impedance.

This may include one or more impedance measurements at a plurality of different frequencies. As discussed above, as ablation of the tissue proceeds, the measured impedance may begin to fall as the tissue properties change. However, since the measured impedance may drop at approximately the same rate for the measurements obtained at both a lower frequency (for example, 46 kHz) and a higher frequency (for example, 460 kHz), the difference in the impedance measured at the low and high frequencies will still reflect the proportion of the ablation electrode 24 in intimate contact with the tissue. In other words, the difference between the impedance measured at the lower frequency and the impedance measured at the higher frequency would drop in a linear manner such that the clinician could accurately ascertain or otherwise verify that the desired contact between the ablation electrode 24 and the target tissue is maintained. Should the drop in impendence begin to stray from the linear relationship, the clinician may be alerted to this observation and may be made aware that the desired contact between the ablation electrode 24 and the target tissue may be lost. In contrast, if impedance was monitored at only a single frequency, the clinician may still observe a drop in impedance but may not be able to readily determine if the drop in impedance was due to ablation of the target tissue or if the drop in impedance was due to loss of contact between the ablation electrode 24 and the target tissue.

The algorithm for measuring the impedance at different frequencies and for determining if the electrode 24 to tissue contact is sufficient may be stored in the memory of a processing unit. In some instances, the processing unit may be the mapping processor 16 or the RF generator 18. The ablation electrode 24 may be in electrical communication with the processing unit such that the impedance can be measured. In some instances, the system 10 may be able to automatically give an additional indication of contact quality by looking at the frequency content of the impedance difference at 46-460 kHz (for example, between 40-120 kHz). This may reduce the problem of excessive filtering showing an impedance difference corresponding to 40% electrode surface contact, which may occur by averaging 80% contact and 0% contact due to a tip completely bouncing off the heart and thus moving from full contact to no contact.

The ablation electrode 24 may be electrically connected to the processing unit including the algorithm in a number of different ways. For example, the electrodes 24 may be connected to the processing unit such that the system 10 uses time-division multiplexing (TDM) to obtain the impedance measurements at two or more frequencies. For example, the system 10 may use sub-channels to switch between measuring the impedance at two or more frequencies. The system 10 may then compute the difference in measured impedance between the two or more frequencies. In another illustrative embodiment, the ablation electrode 24 may be connected to the processing unit such that the system uses frequency-division multiplexing (FDM) to obtain the impedance measurements at two or more frequencies. For example, the system 10 may measure the impedance at multiple frequencies simultaneously and subsequently separate the frequencies via filtering in order to compute the difference in measured impedance.

Those skilled in the art will recognize that the present invention may be manifested in a variety of forms other than the specific embodiments described and contemplated herein. Accordingly, departure in form and detail may be made without departing from the scope and spirit of the present invention as described in the appended claims.

What is claimed is:

1. An ablation device for treating body tissue, comprising:
a catheter having a proximal end region and a distal end region;
an electrode disposed adjacent to the distal end region of the catheter;
a processing unit having a memory, the processing unit in electrical communication with the electrode;
wherein the processing unit is capable of determining a degree of contact between the electrode and a target tissue.

2. The ablation device of claim 1, wherein the processing unit monitors impedance at a plurality of different frequencies.

3. The ablation device of claim 2, wherein the processing unit calculates the difference between the impedance monitored at a first electrode at a first frequency from the impedance monitored at the first electrode at a second frequency.

4. The ablation device of claim 3, wherein during an ablation procedure, the processing unit is configured to monitor the change in impedance to determine if the change in impedance follows a substantially linear relationship to determine the degree of contact between the electrode and the target tissue.

5. The ablation device of claim 1, wherein the electrode includes an ablation electrode.

6. The ablation device of claim 5, further comprising one or more mapping electrodes coupled to the catheter.

7. The ablation device of claim 1, wherein the processing unit includes a mapping processor.

8. The ablation device of claim 1, wherein the processing unit includes an RF generator.

9. A method for ablating a target tissue, the method comprising:
advancing an ablation device through a body lumen to a position adjacent to a target tissue, the ablation device including a catheter shaft having an ablation electrode coupled to the catheter shaft;
measuring the impedance at the target tissue at a plurality of different frequencies including a first frequency and a second frequency;
determining a first difference between the impedance measured at the first frequency and the impedance measured at the second frequency;
ablating the target tissue with the ablation electrode;
during or after ablating the target tissue, determining a plurality of additional differences between the impedance measured at the first frequency and the impedance measured at the second frequency; and
comparing the first difference between the impedance measured at the first frequency and the impedance measured at the second frequency with the plurality of additional differences between the impedance measured at the first frequency and the impedance measured at the second frequency so as to determine the degree of contact between the ablation electrode and the target tissue.

10. The method of claim 9, wherein the first frequency, the second frequency, or both is approximately 46-460 kHz.

11. The method of claim 9, wherein the first frequency is approximately 46 kHz.

12. The method of claim 11, wherein the second frequency is approximately 460 kHz.

13. The method of claim 9, wherein the ablation device is coupled to a processing unit, and wherein the processing unit compares the first difference in impedance with the plurality of additional differences.

14. The method of claim 13, wherein the processing unit includes an RF generator.

15. The method of claim 9, wherein comparing the first difference between the impedance measured at the first frequency and the impedance measured at the second frequency with the plurality of additional differences between the impedance measured at the first frequency and the impedance measured at the second frequency so as to determine the degree of contact between the ablation electrode and the target tissue includes determining if the first difference and the plurality of differences follow a substantially linear relationship.

16. The method of claim 9, wherein comparing the first difference between the impedance measured at the first frequency and the impedance measured at the second frequency with the plurality of additional differences between the impedance measured at the first frequency and the impedance measured at the second frequency so as to determine the degree of contact between the ablation electrode and the target tissue includes determining if the impedance drops at substantially the same rate at both the first frequency and at the second frequency.

17. The method of claim 9, wherein the ablation device includes a display unit with a colored indication of approximate contact between the ablation electrode and the target tissue.

18. The method of claim 9, further comprising measuring the impedance at the target tissue at a third frequency different from both the first frequency and the second frequency.

19. The method of claim 9, wherein the target tissue is a cardiac tissue.

20. An ablation device for treating body tissue, comprising:
a catheter having a proximal end region and a distal end region;
an electrode disposed adjacent to the distal end region of the catheter;
a processing unit having a memory, the processing unit in electrical communication with the electrode;
wherein the processing unit is capable of determining a degree of contact between the electrode and a target tissue by monitoring the change in impedance at two different frequencies and determining if the impedance drops at the same rate at both frequencies.
